(19) United States
(12) Patent Application Publication
(10) Pub. No.: US 2015/0225476 A1
(43) Pub. Date: Aug. 13, 2015

(54) METHOD FOR TREATING A DISEASE OR DISORDER OF THE LUNG BY INHIBITION OF THE HEDGEHOG PATHWAY

(71) Applicant: AGENCY FOR SCIENCE, TECHNOLOGY AND RESEARCH, Connexis, Singapore (SG)

(72) Inventors: Maria Alicia Curotto de Lafaille, Singapore (SG); Victor De Vries, Singapore (SG)

(21) Appl. No.: 14/430,104

(22) PCT Filed: Sep. 23, 2013
(86) PCT No.: PCT/SG2013/000415

§ 371 Date: Mar. 20, 2015

(30) Foreign Application Priority Data
Sep. 21, 2012 (SG) 201207025-6

(51) Int. Cl. C07K 16/18; A61K 39/395; A61K 45/06

(52) U.S. Cl. CPC C07K 16/18; A61K 45/06; A61K 39/3955; C07K 2317/76; A61K 2039/505

(57) ABSTRACT

The present invention is directed to a method of treating a disease or disorder characterised by one or more of the following: decreased lung function, bronchial hyper-responsiveness, hypersecretion of mucus, epithelial cell hyperplasia, smooth muscle hypertrophy, fibrosis or inflammation with an antagonist of a Hedgehog protein, an antagonist of Smoothened, or an antagonist of Gli. The present invention further provides kits for implementing the above method and the use of an antagonist of a Hedge-hog protein, an antagonist of Smoothened, or an antagonist of Gli for treating the above disease or disorder.

### CROSS-REFERENCE TO RELATED APPLICATIONS

This application claims the benefit of priority of Singapore patent application No 201207025-6, filed 21 Sep. 2012 the contents of it being hereby incorporated by reference in its entirety for all purposes.

### TECHNICAL FIELD

The present invention relates to the inhibition or neutralization of the hedgehog (HH) pathways employed as a preventive, or as therapeutic treatment of established disease, such as asthma or chronic obstructive pulmonary disease (COPD), by restoring lung function, decreasing tissue remodeling, decreasing allergic inflammation of the pulmonary system or a combination thereof. More specifically, the present invention relates to the use of an antagonist of a Hedgehog protein, an antagonist of Smoothened, or an antagonist of Gli in order to treat a disease or disorder characterised by one or more of hypersecretion of mucus, epithelial cell hyperplasia, smooth muscle hypertrophy, fibrosis or inflammation, bronchial hyper-responsiveness or decreased lung function.

### BACKGROUND

The hedgehog (HH) pathway is an evolutionary conserved signaling pathway involved in development across metazoan organisms (C. C. Hui and S. Angers, Gli proteins in development and disease. Annu Rev Cell Dev Bio 127, 513-537 (2011); P. W. Ingham et al., Mechanisms and functions of Hedgehog signaling across the metazoa. Nat Rev Genet 12, 393-406 (2011)). HH proteins bind to the membrane receptor patch (PTCH) that in turn inhibits the activity of smoothened (SMO), a heptahelical transmembrane GPCR protein that is essential for the signaling response to HH. HH binding inhibits PTCH activity, allowing activation of SMO in the processing of transcription factors of the GLI family that ultimately decreases the repressor forms of and increases the activator forms of GLI. HH signaling regulates the balance between transcriptional activation and repression by GLI factors, whereby activation of the HH pathway results in transcription of a number of target genes, among them Gli1, Patch and the hedgehog-interacting protein (HHIP). HHIP is a negative regulator that interacts with all three HH genes and attenuates signaling.

The HH pathway is one of several signaling pathways involved in lung development (W. V. Cardoso and J. Lu, Regulation of early lung morphogenesis: questions, facts and controversies. Development 133, 1611-1624 (2006); E. E. Morrisey and B. L. Hogan, Preparing for the first breath: genetic and cellular mechanisms in lung development. Dev Cell 18, 8-23 (2010)). In the developing lung sonic hedgehog (SHH) is secreted by the budding endoderm and signals to the adjacent mesoderm to regulate branching morphogenesis. A high concentration of SHH in the tip of the bud induces HHIP in the mesoderm cells and the suppressive activity of SHH on FGF10 production is attenuated in mesodermal cells, allowing budding to continue. In contrast, low SHH production in the stalks of the bud does not induce HHIP and inhibits FGF10 secretion by the mesoderm and thus lateral budding occurs.

In addition, SHH and other molecules secreted by endodermal cells during development signal to mesodermal progenitors and the mesothelium to affect their differentiation into cartilage, bronchial and vascular smooth muscle cells. SHH regulated expression of FOXF1 is required for smooth muscle and cartilage development. It is known that mice deficient in Shh have foregut developmental defects, tracheao-esophageal fistula, tracheal and lung abnormalities and lack Of airway smooth muscle cells (Y. Litingtung, et al., Sonic hedgehog is essential to foregut development. Nat Genet 20, 58-61 (1998); C. V. Pepicelli et al. Sonic hedgehog regulates branching morphogenesis in the mammalian lung. Curr Biol 8; 1083-1086 (1998)).

Asthma is an obstructive inflammatory airway disease characterized by an exacerbated bronchial contractile response, increased mucus production, sub-epithelial fibrosis and increased smooth muscle mass (S. Al-Muhsen, et al., Remodeling in asthma. J Allergy Clin Immunol 128, 451-462; quiz 463-454 (2011)). The immunopathology of asthma is driven by a Th2-biased immune response, production of IgE antibodies, and mast cell and eosinophilic inflammation. Asthma can manifest as mild, moderate or severe disease, with variable response to standard corticosteroid treatment. However, resistance to corticosteroid treatment is observed in several lung inflammatory diseases including severe asthma and chronic obstructive pulmonary disease (COPD).

The respiratory epithelium is severely affected in asthma, wherein the loss of barrier function, increased differentiation of epithelial cells into Goblet cells and reduction of ciliated cells are some of the alterations of epithelium in asthma. In addition, during stress, injury and inflammation, epithelial cells can secrete cytokines, chemokines and growth factors that affect other tissue cells as well as immune cells in the lung (S. T. Holgate, Epithelium dysfunction in asthma. J Allergy Clin Immunol 120, 1233-1244; quiz 1245-1236 (2007); L. Ramakrishna et al, Cross-roads in the lung: immune cells and tissue interactions as determinants of allergic asthma. Immunol Res, (2012); Hammad, H., and B. N. Lambrecht. Dendritic cells and epithelial cells: linking innate and adaptive immunity in asthma. Nat Rev Immunol 8:193-204 (2008)).

There is currently no cure for asthma, and there is a need for new therapies that target tissue pathology rather than just inflammation, and that can have in impact on severe and corticosteroid resistant asthma.

It is an aim of the present invention to provide therapies for restoring lung function, improve repair, responses and decrease allergic inflammation ultimately resulting in the treatment of asthma, COPD and associated conditions and/or symptoms thereof.

### SUMMARY

According to a first aspect, there is provided a method of treating a disease or disorder characterised by one or more of hypersecretion of mucus, epithelial cell hyperplasia, smooth muscle hypertrophy, fibrosis or inflammation, bronchial hyper-responsiveness or decreased lung function, comprising administering an antagonist of a Hedgehog protein, an antagonist of Smoothened, or an antagonist of Glito a patient in need thereof.

According to the second aspect, there is provided a kit when used in accordance with the above method comprising an antagonist of a Hedgehog protein, an antagonist of Smoothened, or an antagonist of Gli together with instructions for use.

According to a third aspect, there is provided an antagonist of a Hedgehog protein, an antagonist of Smoothened, or an antagonist of Gli for use in the manufacture of a medicament for treating disease or disorder characterised by one or more of hypersecretion of mucus., epithelial cell hyperplasia, smooth muscle hypertrophy, fibrosis or inflammation, bronchial hyper-responsiveness or decreased lung function.

### DEFINITIONS

The following words and terms used herein shall have the meaning indicated:

The term “agonist” as used herein, refers to any molecule which enhances the biological activity of its target molecule. The term “antagonist” as used herein, refers to any molecule that counteracts or inhibits the biological activity of its target molecule. The agonists or antagonists may include but are not limited to peptides, antibodies, or small molecules that bind to their specified target or the targets natural ligand and modulate the biological activity. Non-limiting examples of agonists and antagonists in the context of the present invention include beta-adrenoceptor agonists, adrenergic agonists, long acting beta-adrenoceptor agonists, leukotriene antagonists, an antagonist of Smoothened, an antagonist of Smoothened activation, or an antagonist of Gli. The term “smoothened” refers to the Smoothened (Smo) receptors and non-classical G-protein-coupled receptors that belong to the Frizzled family.

The term “antibody” is used herein in the broadest sense to refer to molecules with an immunoglobulin-like domain and includes monoclonal, recombinant, polyclonal, chimeric, humanised, human, bispecific, multispecific and heteroconjugate antibodies; a single variable domain, a domain antibody, antigen binding fragments, immunologically effective fragments, single chain Fv, diabodies, Tandabs™. Non-limiting examples of antibodies used in the context of the present invention are monoclonal antibody 5E1, anti-IgE antibodies and anti-cytokine antibodies.

The phrase “single variable domain” refers to an antigen binding protein variable domain (for example, V_{H}, V_{HH}, V_{L}) or antigen binding fragment that specifically binds an antigen or epitope independently of a different variable region or domain.

The term “specifically binds” as used herein refers to the antigen binding protein or fragment binding to a target epitope on HH with a greater affinity than that which results when bound to a non-target epitope. Specific binding refers to binding to a target with an affinity that is at least 10, 50, 100, 250, 500, or 1000 times greater than the affinity for a non-target epitope. For example, binding affinity may be as measured by routine methods, e.g., by competition ELISA or by measurement of Kd with BIACORE™, KINEXA™ or PROTEON™.

A “domain antibody” or “dAb” may be considered the same as a “single variable domain” which is capable of binding to an antigen. A single variable domain may be a human antibody variable domain, but also includes single antibody variable domains from other species such as rodent, nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are considered to be “domain antibodies”. As used herein V_{H} includes camelid V_{HH} domains.

As used herein the term “domain” refers to a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A domain can bind an antigen or epitope independently of a different variable region or domain.

As used herein the term “antigen” refers to a molecule that is capable of being bound to by specific antibodies. Antibody-antigen binding is mediated by the sum of many interactions between the antigen and antibody including, for example, hydrogen bonds, van der Waals forces, and ionic and/or hydrophobic interactions. An antigen binds to the complementarity regions on an antibody. The corresponding region(s) of the antigen is referred to as an “antigenic determinant” or “epitope”. Antigens include molecules such as, for example, polypeptides, polynucleotides, carbohydrates, haptens, and the like, from sources such as, for example, plants, animals, viruses, microorganisms, and the like. Antigens also can include substances such as toxins, chemicals, drugs, foreign particles, and the like. For example, in the context of the present invention the antigen may be any molecule that interacts in the HH signaling pathway and is essential for the signaling response to HH, such as HH, PTCH receptor, SMO or GLI.

An antigen binding fragment may be provided by means of arrangement of one or more CDRs on non-antibody protein scaffolds such as a domain. The domain may be a domain antibody or may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroE1 and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than its natural ligand.

An antigen binding fragment or an immunologically effective fragment may comprise partial heavy or light chain variable sequences. Fragments are at least 5, 6, 8 or 10 amino acids in length. Alternatively the fragments are at least 15, at least 20, at least 50, at least 75, or at least 100 amino acids in length.

The term “monoclonal antibody” is used herein as its conventional meaning in referring to a mono-specific antibody secreted by a hybridoma clone. A non-limiting example is the monoclonal antibody 5E1 obtained from the Developmental studies hybridoma cell bank, University of Iowa where the amino acid sequence and structure of monoclonal antibody 5E1 are well characterized and readily available to the public.

The term “small molecule” is used herein to refer to analogs that structurally resembles an antagonist of a Hedgehog protein, an antagonist of Smoothened, or an antagonist of Gli but which has been modified in a targeted and controlled manner. Compared to the starting antagonists or agonists, a small molecule may exhibit the same, similar, or improved utility in modulating HH mediated signaling. Synthesis and screening of small molecules, to identify variants of known compounds, antibodies or the like having improved traits (such as higher binding affinity, or higher selectivity of binding to a target and lower activity levels to non-target molecules) is an approach that is well known in the art. In reference to a small molecule “capable of preventing the binding of Hedgehog to its receptor” as described herein, we refer to any small molecule as defined herein that binds to either of the hedgehog or the associated receptor, Patched, resulting in the modulation of the receptor signaling response to HH.

The term “natural compound” is used herein to refer to a chemical substance produced by a living organism or a chemical substance found in nature that has distinctive pharmacological effects. Such a substance is considered a natural product even if it can be prepared by total synthesis.

The term “neutralize” as used herein in the context of HH neutralization means that the biological activity of HH is reduced or inhibited in the presence of the medicament as described herein in comparison to the activity and expression of HH in the absence of the medicament. The reduction or inhibition in biological activity may be partial or total. Neutralization may be determined or measured using one or more assays known to the skilled person or as described herein. The term “inhibition” as used herein refers to the reduction of a molecule, a reaction, an interaction, a gene, an mRNA, and/or a protein's expression, stability, function or activity by a measurable amount or to prevent entirely. Inhibitors are compounds that, may bind to, partially or totally block stimulation, decrease, prevent, delay activation, inactivate, desensitize, or down regulate a protein, a gene, and mRNA stability, expression, function and activity, such as antagonists.

The term “response” as used herein in the context of corticosteroid treatment, refers to a patient's “responsiveness” or being “responsive” to the pharmacological effects of corticosteroid treatment, and the patient's clinical response to a treatment can include a complete response with evaluable but non-measurable disease or disorder. On the other hand, it can also mean a partial response that is anything less than a complete response. On the other hand, it can also mean a non-response where evidence of disease has remained constant or has progressed.

The terms “therapeutic agent” and “medicament” are used interchangeably herein to refer to a wide variety of substances that, when administered to an organism (human or animal), induce a desired pharmacologic or biological effect, such as a reduction in inflammation.

The term “mucus hypersecretion” as used herein, refers to the excessive production of mucus by the lung epithelial cells, and is a major clinical and pathological feature of asthma, in addition to other conditions, for example cystic fibrosis related bronchiectasis, non-CF bronchiectasis, and chronic obstructive pulmonary disease.

The term “hyperplasia” as used herein, refers to an increased production or proliferation of cells in an organ or tissue, for example the increased accumulation of epithelial or goblet cells within the lung.

The term “hypertrophy” as used herein, refers to the non-tumorous enlargement of an organ or a tissue as a result of an increase in the size rather than the number of constituent cells, for example “smooth muscle hypertrophy” refers to the abnormal enlargement of smooth muscle fibres that in the pulmonary system can narrow the airways and increase reactivity to allergens, infections, irritants, parasympathetic stimulation and other bronchiorestrictive triggers.

The term “inflammation” as used herein refers to non-infectious inflammatory conditions, but can also relate to all the infectious diseases and conditions known to those skilled in the art, where an increase of inflammation is expected. A non-limiting example of inflammation is eosinophilic inflammation. In the context of the present disclosure the inflammation is measurable by analyzing cellular infiltrates and cytokine levels in biological samples. Non-limiting examples of inflammation markers measurable include but are not limited to Transforming growth Factor β (TGF β), T Helper cells cytokines (Th2), Tumor Necrosis Factor-α (TNF-α), interleukin-1 (IL-1), interleukin-2 (IL-2), interleukin-4 (IL-4), interleukin-5 (IL-5), interleukin-6 (IL-6), immunoglobulin E (IgE), immunoglobulin G (IgG), interleukin-13 (IL-13), interleukin-33 (IL-33), and interferon-γ (IFN-γ).

The term “bronchial hyperesponsiveness” (BHR) as used herein, refers to an exaggerated bronchial constriction stimulated by non-specific provocation, and is commonly associated with pulmonary diseases or disorders, for example asthma. “bronchial hyperesponsiveness” can be interpreted solely as bronchoconstriction and hence also encompasses “bronchospasm”, wherein a spasm is a sudden, violent, involuntary contraction of a muscle or a group of muscles.

The term “bronchiolitis” as used herein refers to its conventional meaning of bronchiolar damage induced by acute inhalation exposure to gases, for example anhydrous ammonia, or by infectious agents or other insults to the lower respiratory tract.

The term “asthma” as used herein, refers to is an obstructive inflammatory airway disease characterized by an exacerbated bronchial contractile response, increased mucus production, sub-epithelial fibrosis and increased smooth muscle mass. “Asthma” is classified into 4 main categories of mild intermittent, mild persistent, moderate persistent and severe persistent.

The term “chronic obstructive pulmonary disease” (COPD), as used herein, refers to a partially reversible airflow obstruction caused by an abnormal inflammatory response to toxins, such as cigarette smoke, and can be further divided into chronic obstructive bronchitis and emphysema. Symptoms include but not limited to productive cough and dyspnea. “chronic obstructive pulmonary disease” develops from “chronic bronchitis” if spirometric evidence of airflow obstruction develops. “chronic bronchitis” refers to the presence of a productive cough that produces sputum that occurs most days of the month, three months of a year for two years in a row without other underlying disease to explain the cough.

The term “fibrosis” as used herein, refers to formation of excess fibrous connective tissue in an organ or tissue. A Non-limiting example is “pulmonary fibrosis” that is categorized by subplural fibrosis with sites of fibroblast proliferation and dense scarring, alternating with areas of normal lung tissue; or “cystic fibrosis” that is a hereditary disease of the exocrine glands that primarily affects the gastrointestinal and respiratory systems.

The term “lung function” or “pulmonary function” as used herein, refers to measurements of lung resistance and compliance determined using apparatuses well known in pre-clinical studies, including but not limited to the flexyVent™ apparatus. In humans lower lung function may include reversible airflow obstruction, bronchospasm, with symptoms as wheezing, coughing, chest tightness, and shortness of breath. Lung function is usually evaluated by spirometry. Lower lung function may include a decreased in forced expiratory volume in one second (FEV1), and decrease in peak expiratory flow rate.

The term “parenteral administration” as used herein refers to routes of administration other than through the gastrointestinal tract or lungs, and to administering the medicament by such routes. Thus, “parenteral” as used herein includes, for example, intramuscular, intradermal, subcutaneous, intra-articular (i.e. into the joint, which in turn includes intra-synovial, i.e. into the synovial fluid).

The term “local administration” as used herein refers to the administration of the medicament to the skin or mucosa, including the mucosa of the mouth, nasal and sinus cavities, lower respiratory tract, eyes, gastrointestinal tract, bladder, urethra, and vagina. More specifically, “local administration” may refer to intranasal administration or intra-airway administration via inhalation of the medicament. The term “local administration” as used herein encompasses the meaning of “topical administration” and also includes administration to spatially restricted portions of the body, including portions of the skin, muscle, eyes, and other tissues and organs, and combinations of these.

The term “intravenous administration” is used herein in its conventional sense to refer to the administration of the medicament directly into the vein. The term “oral administration” is used herein in its conventional sense to refer to the administration of the medicament via the mouth.

The term “inhalation” as used herein refers to the intake of air to the alveoli. In specific examples, intake can occur by self-administration of a medicament of the invention while inhaling through a nebulizer or other aerosol-delivery device, or by administration via a respirator, e.g., to a patient on a respirator. The term “inhalation” used with respect to a medicament of the invention is synonymous with “pulmonary administration.”

The term “dispersant” as used herein refers to an agent that assists aerosolization or absorption of the medicament in lung tissue, or both. Preferably, the dispersant is pharmaceutically acceptable. As used herein, the modifier “pharmaceutically-acceptable” means approved by a regulatory agency of the federal or a state government or listed in the U.S. Pharmacopoeia or other generally recognized pharmacopoeia for use in animals, and more particularly in humans.

The terms “simultaneously”, “separately” and “sequentially” as used herein refer to the administration regime of the medicament in combination with the administration of a further one or more therapeutic agent. “Simultaneously administered” refers to the medicament and one or more therapeutic agents being administered in a concomitant administration as well as separate administrations, e.g., within about one-hour, preferably within 5-10 minutes or less. “Separately administered” as used herein refers to the medicament and one or more therapeutic agents being administered independently of one another at an interval, for example at an interval of about a day to several weeks or months. The active agents may be administered in either order. “Sequentially administered” as used herein refers to administration of the medicament and one or more therapeutic agents in sequence, for example at an interval or intervals of minutes, hours, days or weeks, and if appropriate the medicament and one or more therapeutic agents may be administered in a regular repeating cycle. In all cases of “simultaneously”, “separately” and “sequentially” administration, the route of administration may be the same or different.

Unless specified otherwise, the terms “comprising” and “comprise”, and grammatical variants thereof, are intended to represent “open” or “inclusive” language such that they include recited elements but also permit inclusion of additional, unrecited elements.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Disclosure of Optional Embodiments

The present disclosure and embodiments relate to the neutralization of HH proteins and pathway with an antagonist of a Hedgehog protein, an antagonist of Smoothened, or an antagonist of Gli that effectively prevents key pathophysiological features associated with asthma, including bronchial hyper-responsiveness (BHR), lung remodeling and inflammation. Exemplary, non-limiting embodiments of the method of treating a disease or disorder characterized by one or more of hypersecretion of mucus, epithelial cell hyperplasia, smooth muscle hypertrophy, fibrosis, inflammation or decreased lung function, will now be disclosed.

In one embodiment, the Hedgehog protein includes but is not limited to either Sonic Hedgehog, Indian Hedgehog and Desert Hedgehog.

In one embodiment, the antagonist is an antigen binding protein, a peptide, protein, natural compound or small molecule antagonist capable of preventing the binding of Hedgehog to its receptor, PTCH. In another embodiment, the antagonist is an antigen binding protein that includes but is not limited to an antibody or an aptamer or a conjugate thereof.

In another embodiment, the small molecule may be a small molecule inhibitor of sonic hedgehog (Shh) protein that blocks Shh signaling, for example a small molecule inhibitor of sonic hedgehog (Shh) protein includes may be robotnikinin (N-[(4-Chlorophenyl)methyl]-2-[(2R,6S,8E)-5,12-dioxo-2-phenyl-1-oxa-4-azacyclododec-8-en-6-yl]acetamide) or derivatives, analogs, or variants thereof.

In another embodiment, the antibody includes but is not limited to a monoclonal antibody, a recombinant antibody, a polyclonal antibody, chimeric, humanised, bispecific antibody, a heteroconjugate, a single variable domain, domain antibody, a single chain Fv, diabodies, or Tandabs™ or a functional antigen binding fragment thereof. In another embodiment, the monoclonal antibody is 5E1 or a monoclonal antibody which binds the same epitope as 5E1.

In one embodiment of the method, the disease or disorder is a respiratory disease selected from the group consisting of asthma, chronic obstructive pulmonary disease, bronchiolitis obliterans, chronic bronchitis, pulmonary fibrosis and cystic fibrosis.

In another embodiment, the disease or disorder is a gastrointestinal or reproductive disease or disorder characterised by one or more of hypersecretion of mucus, epithelial cell hyperplasia, smooth muscle hypertrophy, fibrosis or inflammation.

In another embodiment, the disease or disorder is asthma.

In another embodiment, the antagonist of a Hedgehog protein, an antagonist of Smoothened, or an antagonist of Gli is formulated into a medicament suitable for administration to a patient. Convenient modes of administration of the medicament include injection (subcutaneous, intravenous, etc.), oral administration, inhalation, transdermal application, topical creams or gels or powders, or rectal administration. Depending on the route of administration, the medicament may be coated with a material to protect the medicament from the action of enzymes, acids and other natural conditions which may inactivate the therapeutic activity of the medicament. The medicament may also be administered parenterally or intraperitoneally or by local administration in the airways.

In another embodiment, the route of administration is selected from the group consisting of oral administration, intravenous administration, parenteral administration and local administration, in the airways. In another embodiment, the route of administration may be subcutaneous, intramuscular, inhalation or intranasal administration. In another embodiment, the route of administration is intravenous administration. In another embodiment, the route of administration is inhalation.

Dispersions of the medicament as described herein may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical compositions of the medicament include sterile aqueous solutions (where water soluble) or dispersions and sterile powders for the extemporaneous preparation of sterile injectable solutions or dispersions. Ideally, the composition is stable under the conditions of manufacture and storage and may include a preservative to stabilise the composition against the contaminating action of microorganisms such as bacteria and fungi. In the case of inhalable solutions, the medicament can be delivered as aerosol particles (solid or liquid) that are of respirable size: that is, particles of a size sufficiently small to pass through the mouth and larynx upon inhalation and into the bronchi and alveoli of the lungs. In general, particles ranging from about 1 to 10 microns in size (more particularly, less than about 5 microns in size) are respirable. Medicaments can be formulated to deliver the desired amount of the medicament to the lungs of a patient by inhalation, or to the nasal respiratory epithelium as a topically applied liquid medicament. Liquid aerosols of respirable particles may be administered by any suitable means, such as by nebulizing a liquid composition containing the medicament (e.g., with a jet nebulizer or an ultrasonic nebulizer), and causing the patient to inhale the nebulized composition. Alternatively, patients maintained on a ventilating apparatus can be administered an aerosol of respirable particles by nebulizing the liquid composition and introducing the aerosol into the inspiratory gas stream of the ventilating apparatus.

In the case of injectable solutions, the carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, it may be preferable to include isotonic agents, for example, sugars, polyalcohols such as mannitol, sorbitol, sodium chloride in the composition. Prolonged absorption of the injectable compositions can be brought, about by including in the composition an agent which delays absorption, for example, aluminium monostearate and gelatin. Preferably, the pharmaceutical composition may further include a suitable buffer to minimise acid hydrolysis. Suitable buffer agent agents are well known to those skilled in the art and include, but are not limited to, phosphates, citrates, carbonates and mixtures thereof.

Single or multiple administrations of the pharmaceutical compositions according to the invention may be carried out. One skilled in the art would be able, by routine experimentation, to determine effective, non-toxic dosage levels of the compound and/or composition of the invention and an administration pattern which would be suitable for treating the diseases and/or infections to which the compounds and compositions are applicable.

Further, it will be apparent to one of ordinary skill in the art that the optimal course of treatment, such as the number of doses of the compound or composition of the invention given per day for a defined number of days, can be ascertained using convention course of treatment determination tests.

Generally, an effective dosage per 24 hours may be in the range of about 0.0001 mg to about 1000 mg per kg body weight; suitably, about 0.001 mg to about 750 mg per kg body weight; about 0.01 mg to about 500 mg per kg body weight; about 0.1 mg to about 500 mg per kg body weight; about 0.1 mg to about 250 mg per kg body weight; or about 1.0 mg to about 250 mg per kg body weight. More suitably, an effective dosage per 24 hours may be in the range of about 1.0 mg to about 200 mg per kg body weight; about 1.0 mg to about 100 mg per kg body weight; about 1.0 mg to about 50 mg per kg body weight; about 1.0 mg to about 25 mg per kg body weight; about 5.0 mg to about 50 mg per kg body weight; about 5.0 mg to about 20 mg per kg body weight; or about 5.0 mg to about 15 mg per kg body weight. In another embodiment, an effective dosage per 24 hours may be in the range of about 2 to 15 mg per kg body weight.

Alternatively, an effective dosage may be up to about 800 mg/m². For example, generally, an effective dosage is expected to be in the range of about 25 to about 800 mg/m², 25 to about 500 mg/m², about 25 to about 350 mg/m², about 25 to about 300 mg/m², about 25 to about 250 mg/m², about 50 to about 250 mg/m², and about 75 to about 150 mg/m².

In another embodiment, the medicament is administered with one or more further therapeutic agents.

In another embodiment, the one or more further therapeutic agents are selected from the group consisting of short acting beta-adrenoceptor agonists, anticholinergic agents, adrenergic agonists, corticosteroids, long acting beta-adrenoceptor agonists, leukotriene antagonists, an antagonist of Smoothened, an antagonist of Smoothened activation, an antagonist of Gli, anti-IgE antibodies or compounds, anti-cytokine antibodies or compounds and mast cell stabilizers.

In another embodiment, the one or more further therapeutic agents are selected from the group consisting of cyclopamine or derivatives thereof, vismodegib, IPI-926, LDE225, XL139 and PF-0449913.

In another embodiment, the medicament and the one or more further therapeutic agents are administered sequentially, simultaneously or separately.

In another embodiment, the treatment as described herein restores C/EBPα levels in the lung to improve a response to corticosteroid treatment in asthma.

In another embodiment, the treatment increases the expression of C/EBPα in the lung to enhance a response to corticosteroid treatment for asthma.

There is also provided the use of an antagonist of a Hedgehog protein, an antagonist of Smoothened, or an antagonist of Gli in the manufacture of a medicament for treating a disease or disorder characterized by one or more of hypersecretion of mucus, epithelial cell hyperplasia, smooth muscle hypertrophy, fibrosis or inflammation.

In one embodiment, the Hedgehog protein includes but is not limited to either of Sonic Hedgehog, Indian Hedgehog or Desert Hedgehog.

In one embodiment, the antagonist is an antigen binding protein, a peptide, protein, natural compound or small molecule antagonist capable of preventing the binding of Hedgehog to its receptor. In another embodiment, the antagonist is an antigen binding protein that includes but is not limited to an antibody or an aptamer or a conjugate thereof.

In another embodiment, the antibody includes but is not limited to a monoclonal antibody, a recombinant antibody, a polyclonal antibody, chimeric, humanised, bispecific antibody, a heteroconjugate, a single variable domain, domain antibody, a single chain Fv, diabodies, or Tandabs™ or a functional antigen binding fragment thereof.

In another embodiment, the monoclonal antibody is 5E1 or a monoclonal antibody which binds the same epitope as 5E1.

In another embodiment, the disease or disorder is a respiratory disease is selected from the group consisting of asthma, chronic obstructive pulmonary disease, bronchiolitis obliterans, chronic bronchitis, pulmonary fibrosis and cystic fibrosis.

In another embodiment, the disease or disorder is a gastrointestinal or reproductive disease or disorder characterized by one or more of hypersecretion of mucus, epithelial cell hyperplasia, smooth muscle hypertrophy or, fibrosis.

In another embodiment, the disease or disorder is asthma.

In another embodiment, the medicament is to be administered with one or more further therapeutic agents.

In another embodiment, the one or more further therapeutic agents are selected from the group consisting of short acting beta-adrenoceptor agonists, anticholinergic agents, adrenergic agonists, corticosteroids, long acting beta-adrenoceptor agonists, leukotriene antagonists, an antagonist of Smoothened, an antagonist of Smoothened activation, an antagonist of Gli, anti-IgE antibodies or compounds, anti-cytokine antibodies or compounds and mast cell stabilizers.

In another embodiment, the antagonist of Smoothened is selected from the group consisting of cyclopamine((3β,23R)-17,23-Epoxyveratraman-3-ol) or derivatives thereof, vismodegib (2-Chloro-N-(4-chloro-3-pyridin-2-ylphenyl)-4-methylsulfonylbenzamide), IPI-926 (saridegib), LDE225 (Erismodegib; N-(6-((2R,6S)-2,6-dimethylmorpholino)pyridin-3-yl)-2-methyl-4′-(trifluoromethoxy)-[1,1′-biphenyl]-3-carboxamide), XL139 (N-(2-methyl-5-((methylamino)methyl)phenyl)-4-((4-phenylquinazolin-2-yl)amino)benzamide) and PF-0449913 (described as part of National Institute of Health Clinical. Trial Identifier No. NCT00953758).

In another embodiment, the medicament and the one or more further therapeutic agents are administered sequentially, simultaneously or separately.

In another embodiment, said medicament restores C/EBPα levels in the lung to improve a response to corticosteroid treatment in asthma.

In another embodiment, said medicament increases the expression of C/EBPα in the lung to enhance a response to corticosteroid treatment for asthma.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1. Shows a schematic representation of experimental asthma groups in preventive and therapeutic treatments with anti-HH antibody 5E1 or isotype control (left) and end-point analysis (right).

FIG. 2. Shows that the administration of anti-HH antibody 5E1 prevents the activation of the HH pathway in a chronic model of experimental asthma. The results shown are in mean±SEM (Standard error of mean) of the ratio of individual mouse values to the untreated group average (=1). Statistically significant differences are indicated (p values).

FIG. 3. Shows that the inhibition of the HH pathway during induction of experimental asthma suppresses BHR. Results are mean±SEM. The tables show statistic values for two-group comparisons.

FIG. 4. Shows that the inhibition of the HH pathway during induction of experimental asthma suppresses smooth muscle hyperplasia, as demonstrated by Q-PCR analysis of the expression smooth muscle genes (A), and histological quantification of smooth muscle in lung sections stained with anti α-smooth muscle actin antibodies (B and C). Expression of cebpa is also shown (D. The results shown are in mean±SEM of the ratio of individual mouse values to the untreated group average (=1): Statistically significant differences are indicated (p values).

FIG. 5. Shows that the inhibition of the HH pathway prevents epithelial remodeling during induction of experimental asthma. A: Q-PCR analysis of epithelial gene transcripts in total lung RNA from mice after 8 weeks of treatment. Muc5ac (tracheobronchial/gastric mucin 5 subtypes A and C), Sftpc (Pulmonary surfactant-associated protein C, Pro-SpC, airway epithelial cells type II), FoxJ1 (forkhead box protein J1, ciliated cells), Scgb1a1 (uteroglobin, Clara cell 10 KDa secretory protein CC10), Foxa2 (forkhead box A2), Spdef (SAM pointed domain containing ets transcription factor). B: Shows a representative pictures of PAS staining of lung sections from the mice used in A. C: Quantification of PAS+ areas.

FIG. 6. A: Shows that treatment with anti-HH antibody 5E1 prevents the alteration in the expression of extracellular matrix proteins. B and C: Demonstrates that anti-HH treatment results in lower collagen deposition.

FIG. 7. Shows that treatment with anti-HH antibody 5E1 prevents lung inflammation. A: shows the total leukocyte counts BAL infiltrates in mice after 8 weeks of treatment. B: Shows pie charts of the compositional make-up of the leukocytes in the BAL. C and D: Demonstrates decreased infiltration of inflammatory cells in the lung.

FIG. 8. A: Shows that the inhibition of the HH pathway during induction of experimental asthma causes reduced production of TH2 cytokines RNA, and of TGFβ. B: Shows decreased expression of IgE transcripts in the lung of mice treated with anti-HH antibodies.

FIG. 9: Shows that a 4 week preventive treatment with anti-HH antibody prevents expression of Shh and HH-transcriptional target genes in mice treated with OVA or HDM.

FIG. 10: Shows that a 4 week preventive treatment with anti-HH antibodies prevents BHR and reduces leukocyte infiltration in the BAL. Mice were treated for 4 weeks with 100 ug OVA or 20 ug HDM intranasal alone or in combination with either anti-HH antibody 5E1 or isotype control. A: Lung resistance and compliance were determined using a flexyVent™ apparatus (SIREQ). Escalating doses of methacholine were administered by inhalation. Results are mean±SEM. B: Total leukocyte counts in the lungs and BAL of the mice used in A.

FIG. 11: Shows that the preventive treatment with anti-HH antibodies reduces inflammatory infiltrates in the BAL of mice treated with OVA (A) or HDM (B).

FIG. 12: Shows that the preventive treatment with anti-HH antibodies in mice treated with HDM inhibits the production of inflammatory cytokines (A) and IgE (B) in the lung.

FIG. 13: Shows that the preventive treatment with anti-HH antibodies in mice treated with HDM inhibits epithelial activation (A) and increase in smooth muscle mass (B) in the lung.

FIG. 14: Shows that the treatment of established chronic airway inflammation with anti-HH antibody suppresses the activation of the HH pathway in the lung

FIG. 15: Shows that the treatment of established chronic airway inflammation with anti-HH restores lung function as measured by reduced resistance and increase compliance to a methacholin challenge.

FIG. 16: Shows that the treatment of established chronic airway inflammation with anti-HH decreases smooth muscle mass as measured by Q-PCR analysis of the expression of smooth muscle proteins (A) and quantification of a-smooth muscle protein in lung sections (B). Anti-HH treatment results in increased expression of cebpa (C).

FIG. 16: Shows that the treatment of established chronic airway inflammation with anti-HH decreases smooth muscle mass as measured by Q-PCR analysis of the expression of smooth muscle proteins (A) and quantification of a-smooth muscle protein in lung sections (B). Anti-HH treatment results in increased expression of cebpa (C).

FIG. 17: Shows that the treatment of established chronic airway inflammation with anti-HH antibody 5E1 normalizes the expression of epithelial genes (A) and reduces Goblet cell hyperplasia, as measured by the quantification of PAS+ areas in lung sections (B and C).

FIG. 18: Shows that the treatment of established chronic airway inflammation with anti-HH antibody 5E1 suppresses ECM remodeling.

FIG. 19: Shows that the treatment of established chronic airway inflammation with anti-HH suppresses the infiltration of inflammatory cells in the airway lumen (A and B) and reduces cellular inflammation in the lung (C and D).

FIG. 20: Shows that the treatment of established chronic airway inflammation with anti-HH suppresses the production of Th2 cytokines and TGFβ in the lung.

FIG. 21: Demonstrates increased expression of SHH (A-C) and GLI1 (D and E) in lung sections from fatal asthma cases.

### EXAMPLES

Non-limiting examples of the invention, including the best mode, and a comparative example will be further described in greater detail by reference to specific Examples, which should not be construed as in any way limiting the scope of the invention.

The present invention and examples elucidate an essential role for the HH signaling pathway in asthma pathogenesis and the associated symptoms. In the following examples it is demonstrated that the expression of SHH and GLI1 are increased in both human asthma and experimental asthma. Importantly, it is demonstrated that neutralization of the HH pathway represents an attractive preventive or therapeutic treatment of established disease, for restored normal lung function and reduced. Th2 inflammation and tissue remodeling in experimental asthma. Thus, the activation of the HH pathway was found to be essential in establishing and maintaining chronic processes of asthma pathophysiology.

### Example 1

### Increased Activation of the HH Pathway in Experimental Asthma and Prevention of Asthma by Neutralizing HH Proteins

To determine whether the hedgehog pathway is activated in experimental asthma, Chronic experimental asthma was induced in A/J mice by intranasal administration of chicken ovalbumin (OVA) three times a week for 4 or 8 weeks period. Chronic experimental asthma was also induced in BALB/c mice by treatment with house dust mite (HDM) extract three times a week for a period of 4 weeks. These treatments resulted in allergic sensitization to OVA (in A/J mice) and to HDM (in BALE/c mice), increased serum IgE level, eosinophilic inflammation in the lung and the airways, Goblet cell differentiation and increased mucus production, smooth muscle mass increase and BHR.

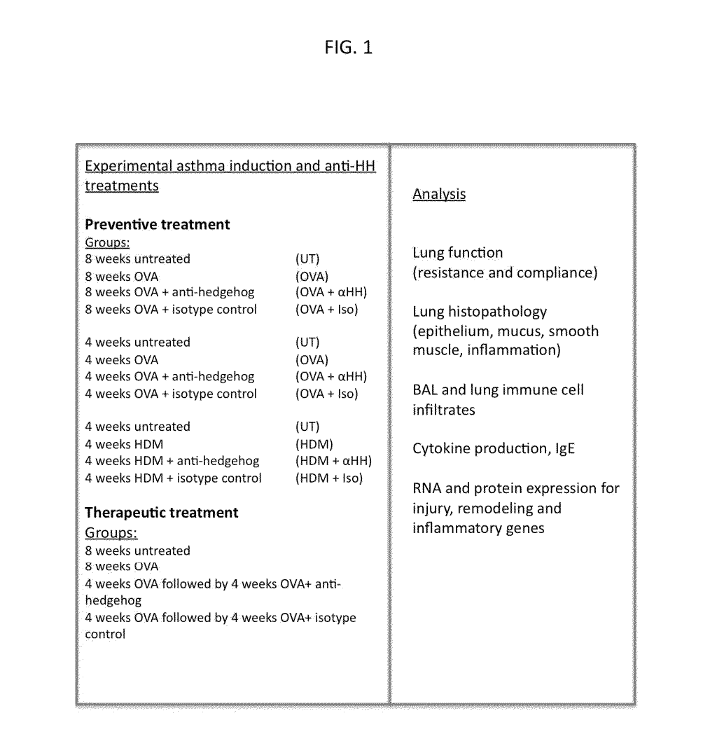

To determine if the inhibition of the HH pathway would affect experimental asthma, the anti-HH antibody 5E1 or an isotype control was administered to mice while they were treated with intranasal OVA or intranasal HDM to induce asthma (preventive treatment) or to mice in which experimental asthma had already been induced (therapeutic treatment). The preventive and therapeutic experiments, mouse groups and end point analysis are described in FIG. 1.

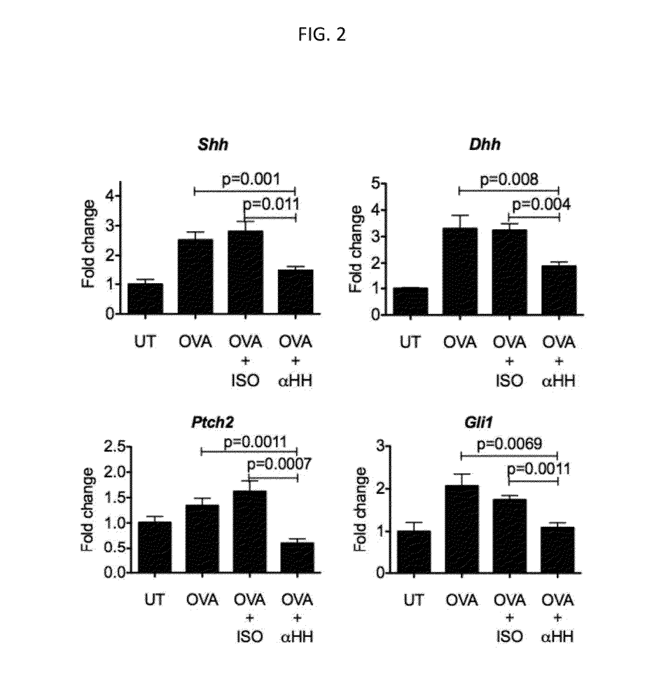

RNA expression analysis of genes of the HH pathway in the lung of untreated or 8-week OVA treated mice treated demonstrated increased expression of Hh genes Shh and Dhh, and of Gli1, a known HH target. These results indicate an upregulation of the HH pathway during experimental asthma. In contrast, it was demonstrated that treatment with the anti-HH antibody 5E1 (a-HH), but not with isotype control antibody (ISO), not only prevented the increased expression of HH-target Gli1, but also resulted in reduced expression of the Hh genes (FIG. 2). Therefore inhibition of the HH pathway decreases the expression of target genes (an expected outcome) but also suppresses the mechanism involved in the upregulation of the Hh genes transcription.

The following findings support that inhibition of the HH pathway prevented the development of BHR, epithelial remodeling, production of mucus, smooth muscle mass increase, production of Th2 cytokines, and infiltration of inflammatory cells in the airways. Thus, targeting the HH pathway was shown to prevent the development of all major pathophysiological features of asthma.

### Inhibition of the HH Pathway Prevents Development of BHR

Lung function was measured in mice after 8 weeks of preventive treatment with anti-HH antibody 5E1 by administration of escalating doses of the bronchoconstrictor drug methacholine delivered through inhalation to artificially ventilated mice. Drug delivery, ventilation and lung resistance and compliance measurements were performed using a flexyVent apparatus (used to assess lung function in mice). An increase airflow resistance indicates excessive narrowing of the airways, while decreased compliance points to a loss of distensibility due to increased lung rigidity.

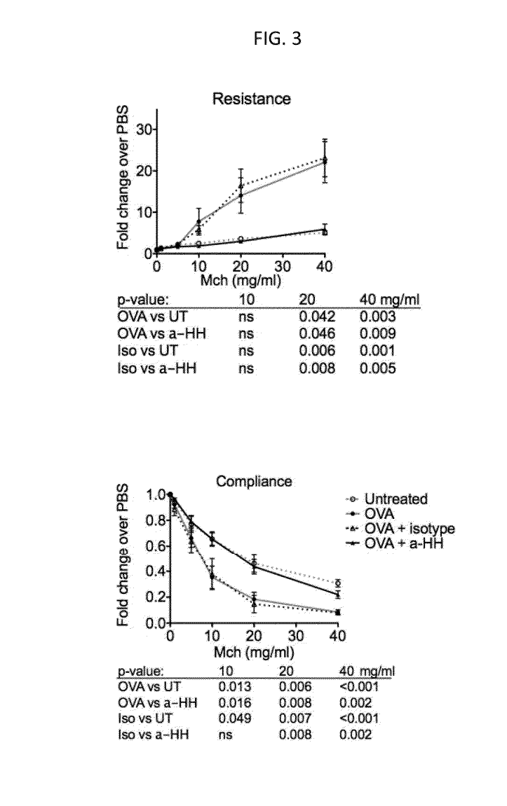

Mice treated with OVA alone or OVA+isotype control antibody had significantly higher lung resistance and decreased lung compliance in response to increased doses of inhaled methacholine than untreated mice (FIG. 3). In contrast, mice treated with OVA+ anti-HH antibody 5E1 behaved as consistent with the untreated mice. Thus, inhibition of the HH pathway during induction of experimental asthma in mice prevented the development of BHR.

### Inhibition of the HH Pathway Prevents the Increase in Smooth Muscle Mass

Bronchial smooth muscle remodeling is a distinct tissue feature of asthma, and its main characteristic is an increase in the thickness (mass) of the smooth muscle bundles (S. Al-Muhsen, J. R. Johnson, Q. Hamid, Remodeling in asthma. J Allergy Clin Immunol 128, 451-462; quiz 463-454 (2011); Halayko, A. J. et al. Airway smooth muscle phenotype and function: interactions with current asthma therapies. Curr Drug Targets 7:525-540 (2006)). Thermoplasty treatment in severe asthma, which reduces bronchial smooth muscle mass, results in decrease clinical symptoms (Castro, M et al. Persistence of effectiveness of bronchial thermoplasty in patients with severe asthma. Ann Allergy Asthma Immunol 107:65-70 (2011); Cox, G et al. Asthma control during the year after bronchial thermoplasty. N Engl J Med 356:1327-1337 (2007)), an indication that smooth muscle remodeling contributes to asthma severity.

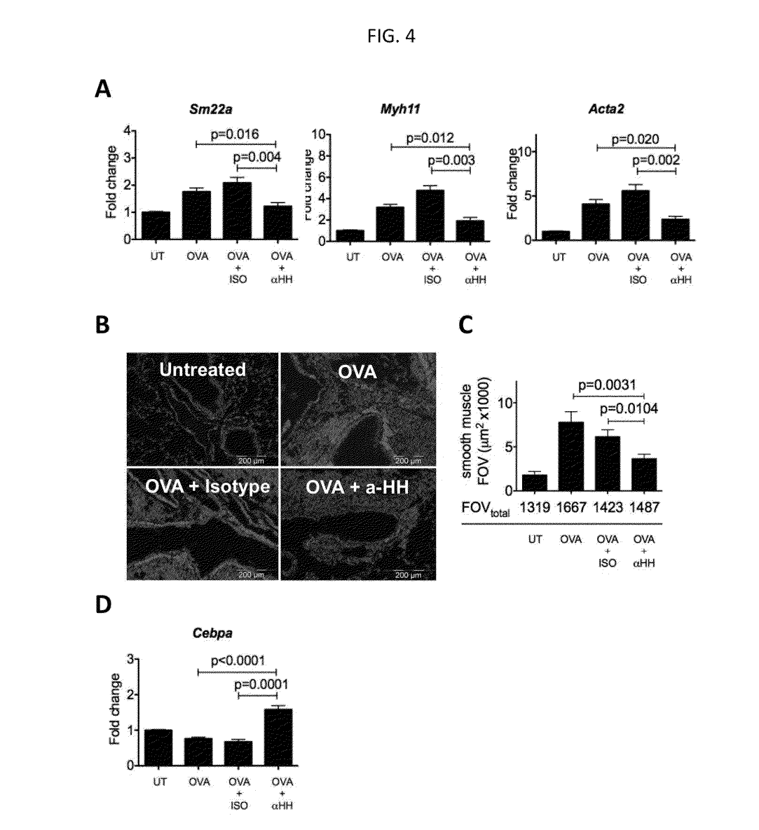

Following the aforesaid 8-week treatment with anti-HH antibody 5E1, the smooth muscle mass was investigated to determine if a reduction had resulted. This was qualified by Q-PCR analysis of the RNA expression of smooth muscle genes α-smooth muscle actin (Acta2), smooth muscle associate protein 22-α (Sm22a) and smooth muscle myosin heavy chain (Myh11) (FIG. 4 A). A marked increase in the expression of smooth muscle proteins RNA was found in OVA and OVA+isotype treated groups relative to the untreated group, consistent with increase smooth muscle mass during experimental asthma. Treatment with anti-HH antibody 5E1 was shown to significantly inhibit the smooth muscle genes over expression. A decrease in smooth muscle mass was also corroborated by the quantification of areas that stained with α-smooth muscle actin antibodies in lung sections (FIGS. 4B and C).

In addition, anti-HH antibody 5E1 treatment resulted in increased expression of cebpa (FIG. 4D), a gene that negatively regulates bronchial smooth muscle cell proliferation. In this regard, the anti-proliferative activity of corticosteroids in lung mesenchymal cells is mediated through the formation of C/EBPα-GC-receptor complexes. C/EBPα is a CCAAT/enhancer binding transcription factor with anti-proliferative effect in several organs including the lung. Lung smooth muscle cells from healthy individuals express C/EBPα and are responsive to corticosteroid. In contrast, smooth muscle cells from asthmatic subjects express reduced levels of C/EBPα as indicated by the decreased expression of cebpa, resulting in the increased proliferation of the cells in vitro compared to cells from healthy subjects, and a poor response to corticosteroids.

These results demonstrate a preventive effect of anti-HH antibody 5E1 administration in smooth muscle pathology during induction of experimental asthma.

### Inhibition of the HH Pathway Reduces Epithelial Cell Activation and Production of Mucus

Epithelial injury with loss of barrier function, loss of ciliated cells, Goblet cell hyperplasia and excess mucus production are characteristics of respiratory epithelial dysfunction in asthma (S. Al-Muhsen et al., Remodeling in asthma. J Allergy Clin Immunol 128, 451-462; quiz 463-454 (2011); S. T. Holgate, Epithelium dysfunction in asthma. J Allergy Clin Immunol 120, 1233-1244; quiz 1245-1236 (2007); L. Ramakrishna et al., Cross-roads in the lung: immune cells and tissue interactions as determinants of allergic asthma. Immunol Res, (2012)). Excess mucus and airway narrowing due to broncho-constriction are mainly contributors to airway occlusion. Differentiation of epithelial cells into Goblet cells in asthma is reflected in a change in epithelial transcription factors expression. Foxa2 is a suppressor of Goblet cell differentiation while Spedf is essential for Goblet cell differentiation. Foxa2 gene expression is reduced while Spdef gene expression is increased in human and experimental asthma. Increase epithelial secretion is reflected by the increased production of surfactants and mucins, such as Pulmonary surfactant-associated protein C (pro-SpC or Sftpc) and Muc5a (L. Ramakrishna, et al., Cross-roads in the lung: immune cells and tissue interactions as determinants of allergic asthma. Immunol Res, (2012).). A reduction in ciliated cells and Clara cells in asthma is indicated by the reduced expression of the transcription factors gene Foxj1 and the Clara cell secretory protein CC10 gene Scgb1a1 respectively.

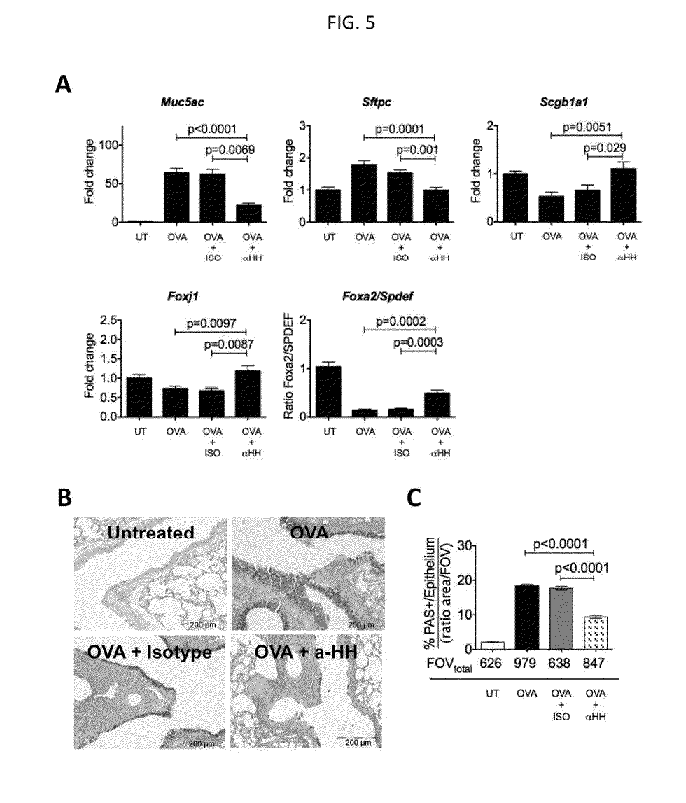

Analysis of the mRNA expression for pro-SPc and Muc5a genes demonstrated an increase in the OVA and OVA+isotype-control groups relative to untreated mice (FIG. 5 A). Inhibition of HH in the OVA+ anti-HH group significantly prevented the increase in pro-SPc and Muc5a transcription. Expression of the transcription factors genes Foxa2 and Spdef corroborated the epithelial activation pattern. Foxa2 expression was reduced in the OVA and OVA+isotype control groups while expression was closer to the untreated mice in the OVA+ anti-HH group.

These results suggest that anti-HH treatment impairs the detrimental effects of OVA treatment on epithelial homeostasis, as revealed by the increased Foxa2/Spdef expression ratio, and increased expression of Scgb1a1 and Foxj1 (FIG. 5 A).

Moreover, goblet cell hyperplasia was analyzed in lung sections by PAS staining. A higher frequency of PAS+ epithelial cells was found in sections from OVA and OVA+isotype-control groups than in sections from the OVA+ anti-HH antibody 5E1 group. Representative sections from untreated and treated mice are shown in FIG. 5 B and quantification of PAS+ areas is shown in FIG. 5 C.

Thus, the above results corroborate that anti-HH treatment during induction of experimental asthma prevents epithelial Goblet cell hyperplasia and the excessive production of mucus while helping to maintain epithelial homeostasis.

### Inhibition of the HH Pathway Prevents Extra Cellular Matrix (ECM) Remodeling

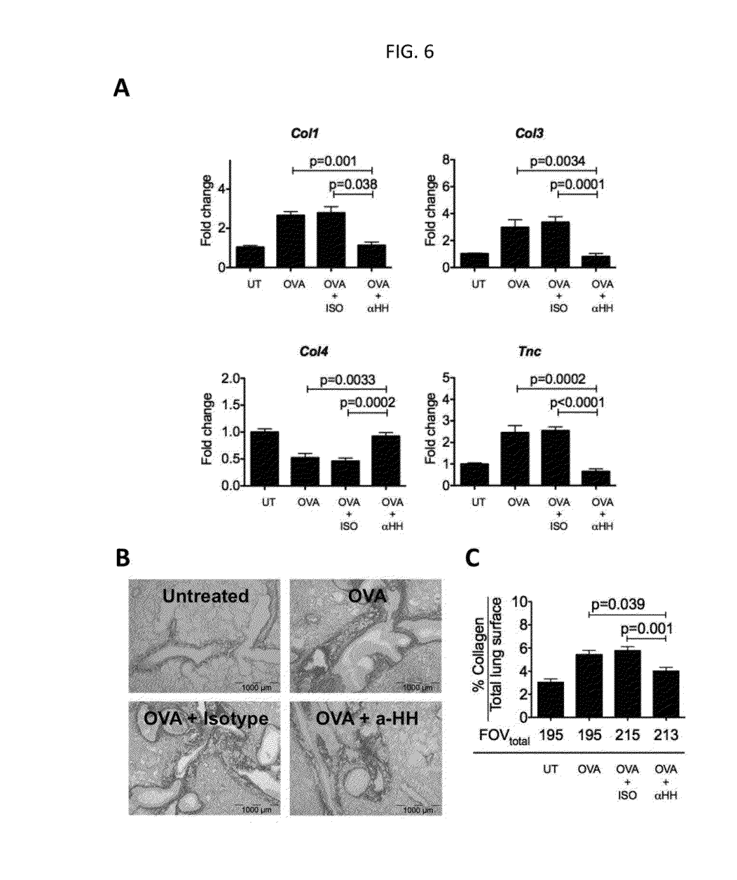

Changes in the composition of the extracellular matrix (ECM) in the lung are a typical feature of asthma (L. Ramakrishna et al., Cross-roads in the lung: immune cells and tissue interactions as determinants of allergic asthma. Immunol Res, (2012).). In concordance with data from human asthma (Burgess, J. K. The role of the extracellular matrix and specific growth factors in the regulation of inflammation and remodelling in asthma. Pharmacol Ther 122:19-29 (2009)), we detected increase expression of collagen I (Col1), collagen III (Col3) and tenascin C (Tnc), and decreased expression of collagen IV (Col4) in our model of chronic asthma, but simultaneous administration of a-HH antibody alongside the OVA treatment blocked these effects (FIG. 6A). Quantification of collagen by picro-sirius red staining of lung sections showed an increase in collagen content in OVA and OVA+Iso groups, which was significantly lower in mice treated with OVA+a-HH (FIGS. 6, B and C). Together, these data demonstrate that the HH signaling pathway impacts on key aspects ECM remodeling in chronic experimental asthma.

### Inhibition of HH Results Prevents the Accumulation Inflammatory Cells in the Airway Lumen

Asthma is a chronic inflammatory disease of the airways and the lung. Inflammation in asthma is mostly found in the walls of the conducting airways, although small airways and the lung parenchyma can also be affected. The presence of inflammatory cells in the airway lumen is also a feature of asthma and lung inflammation in general.

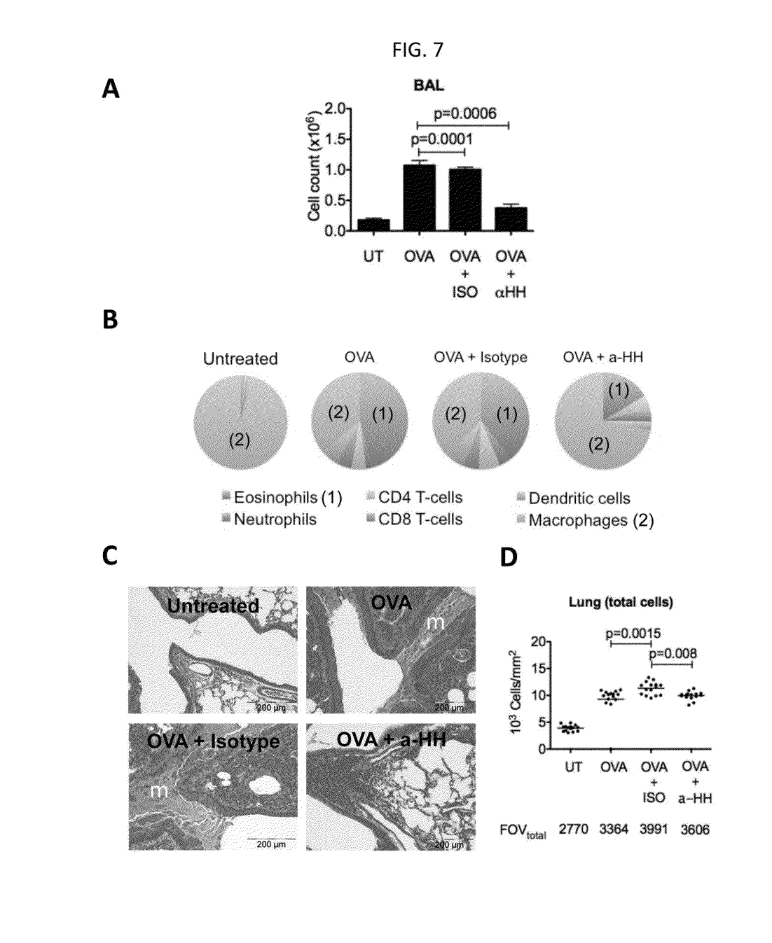

Inflammatory cell numbers were analyzed by flow cytometry in the lungs and broncho-alveolar lavage (BAL) of mice from the 8-week preventive treatment groups. The number of cells infiltrating the lung was higher in all OVA-treated groups than in untreated controls, but did not differ among OVA-treated groups (FIG. 7A). The cell composition of the infiltrated was analyzed by flow cytometry and was found to be of similar composition in all OVA-treated groups.

In contrast, analysis of the BAL showed significant reduction of inflammatory cells in the OVA+ anti-HH antibody 5E1 group when compared with the OVA and OVA+isotype groups (FIG. 7A). All inflammatory cell populations analyzed (eosinophils, neutrophils, dendritic cells, CD4 and CD8 T cells) except macrophages, were reduced in the BAL of OVA+anti-HH antibody 5E1 group compared to OVA and OVA+isotype groups. The largest reduction was in granulocytes eosinophils and neutrophils, while partial reduction was observed in dendritic cells, CD4 and CD8 cells (FIG. 7B). As is well-known in the art, macrophages are the main resident of an immune cell population in the healthy lung, where they play a surveillance role and maintain a suppressive environment through cell-cell interactions with epithelial cells.

There was also a significant reduction in lung inflammatory cellular infiltration in mice treated with a-HH (FIGS. 7, C and D).

Thus, inhibition of the HH pathway greatly prevented the infiltration of inflammatory cells in the airways and lung while maintaining the homeostatic macrophage population.

### Inhibition of HH Decreases the Production of Th2 Inflammatory Cytokines and Inflammatory Mediators.

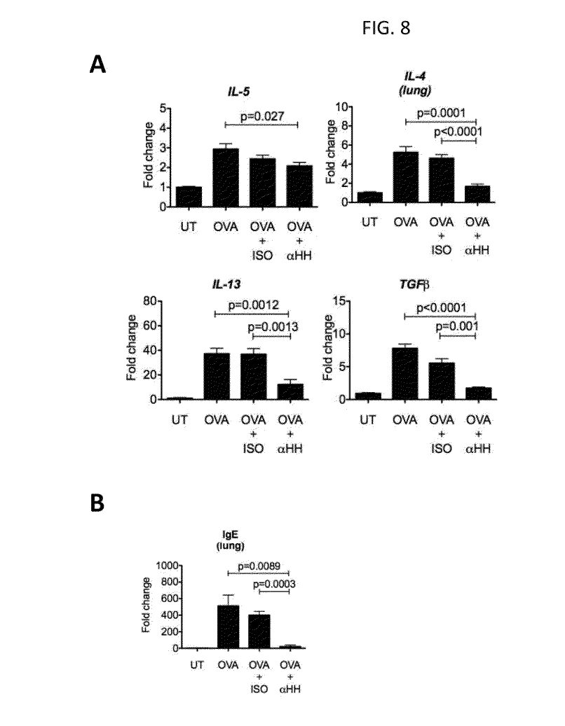

Further analysis of the mRNA expression levels in the 8-week OVA treated mice showed that administration of anti-HH antibody 5E1 during the 8 weeks of OVA-intranasal treatment resulted in a lower mRNA expression of Th2 cytokines IL-4 and IL-13, and of TGFβ (FIG. 8A), and lower expression of IgE (FIG. 8B) in the lung. These results are consistent with reduced in flammatory stimuli in the lung tissue as a consequence of inhibition of HH.

Four Week Preventive Treatment with Anti-HH Antibody 5E1 During Induction of Experimental Asthma

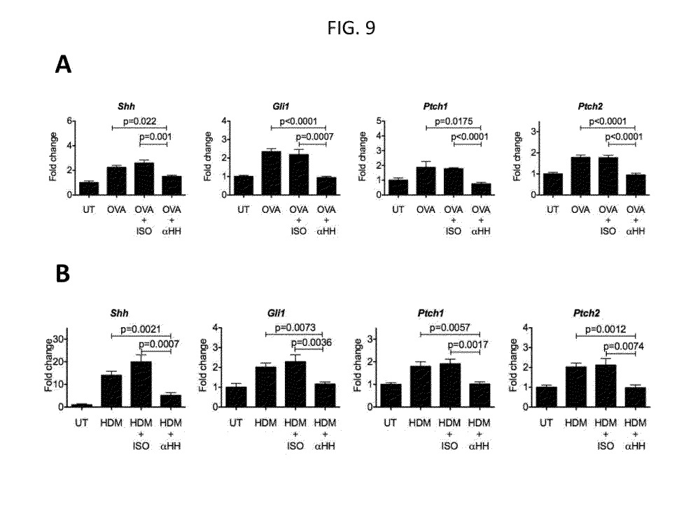

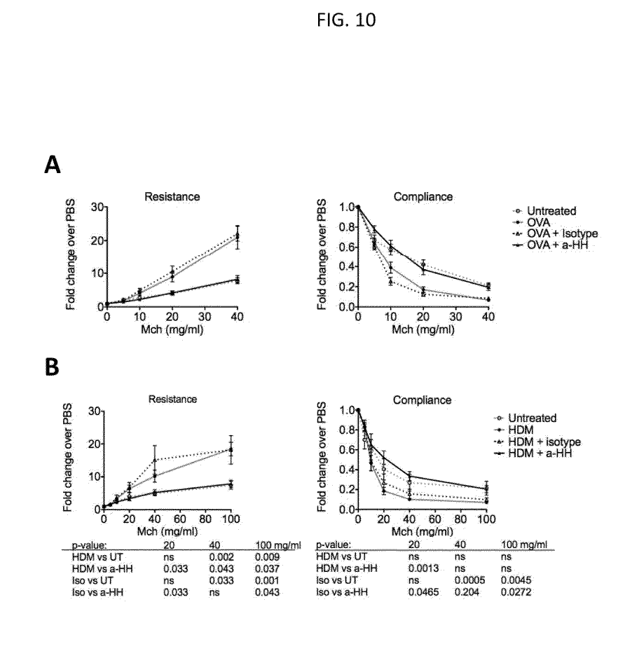

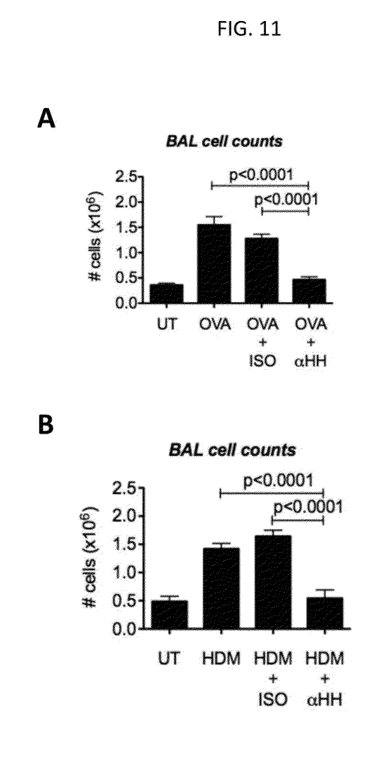

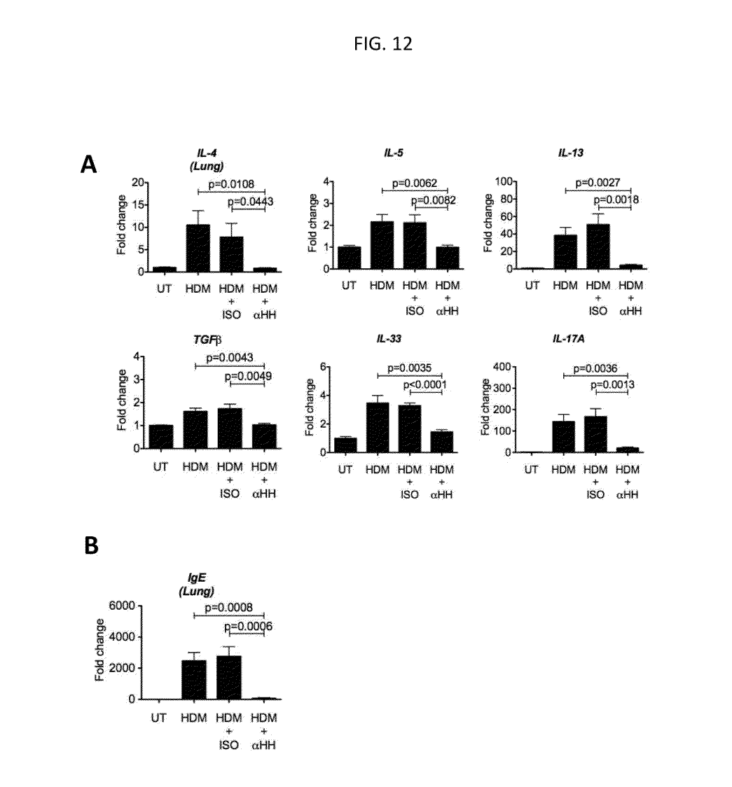

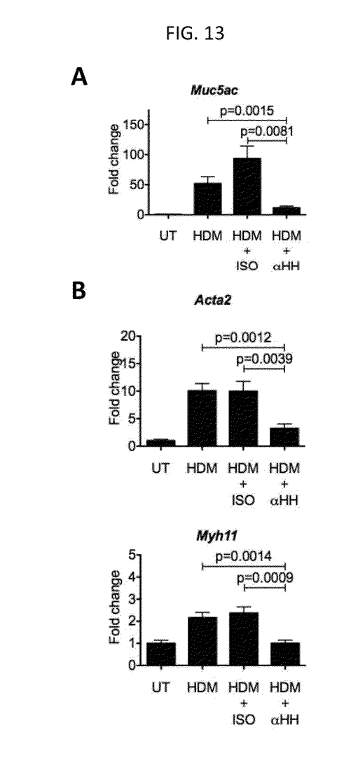

A 4-week preventive treatment with anti-HH antibody 5E1 simultaneously to intranasal OVA administration (FIG. 9A) or simultaneously to HDM administration (FIG. 9B) resulted in reduced expression of Shh and reduced activation of the HH pathway in the lung, as determined by expression of Gli1, Ptch1 and Ptch2. Neutralization of HH proteins prevented development of BHR, as demonstrated by reduced resistance and increased compliance in lung function tests (FIG. 10A:OVA; FIG. 10B:HDM). Similarly to the 8-week treatment, the 4-week preventive treatment prevented the accumulation of inflammatory cells in the BAL. (FIGS. 11 A and B). Neutralization of HH in HDM-induced asthma prevented the expression of cytokines IL-4, IL-5, IL-13, IL-17A, IL-33 and TGFb (FIG. 12A) and the expression of IgE (FIG. 12B) in the lung. Remodeling of epithelium (FIG. 13A), and smooth muscle (FIG. 13B) was also prevented by HH neutralization in HDM-treated mice.

Thus, neutralization of HH proteins prevented loss of lung function, tissue remodeling and inflammation in OVA and HDM models of chronic experimental asthma.

### Example 2

### Therapeutic Benefit Anti-HH Treatment on Established Experimental Asthma

To determine if treatment with anti-HH treatment would reverse established asthma pathology, anti-HH antibody 5E1 or an isotype control were administered to mice 4 weeks after the initiation of the intranasal OVA treatment and for a period of 4 weeks. At the end of the treatment, lung function, lung histopathology and inflammation were analyzed as described in Example 1.

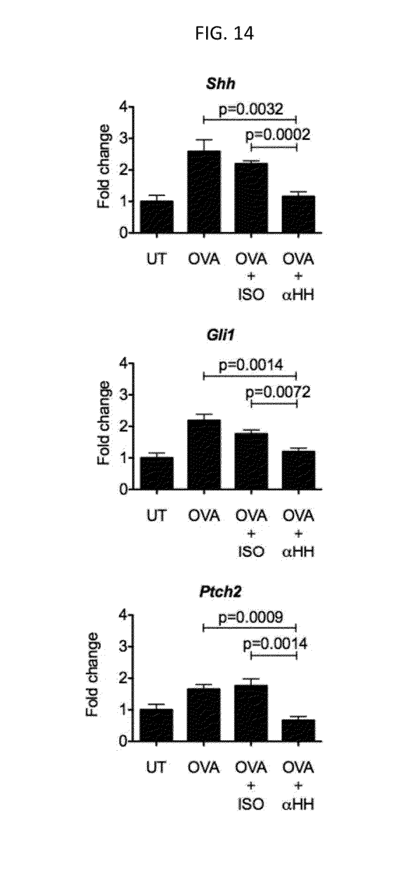

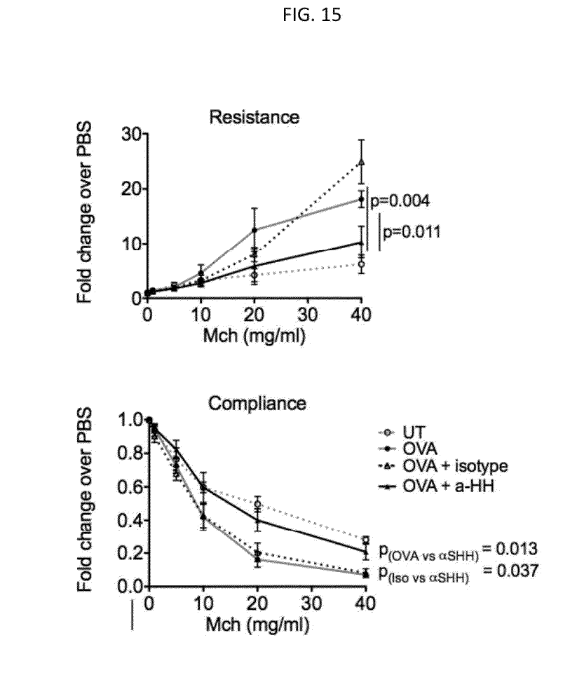

The administration of anti-HH antibody 5E1 was demonstrated to reverse the increased expression of HH pathway genes Shh, Gli1 and Ptch2, indicating a successful inhibition of the HH-pathway as well as the mechanisms involved in higher Shh expression in asthma (FIG. 14). In addition, administration of anti-HH antibody 5E1 was demonstrated to significantly improved lung function, as measured by the decreased resistance and increased compliance response to escalating doses of methacholine (FIG. 15).

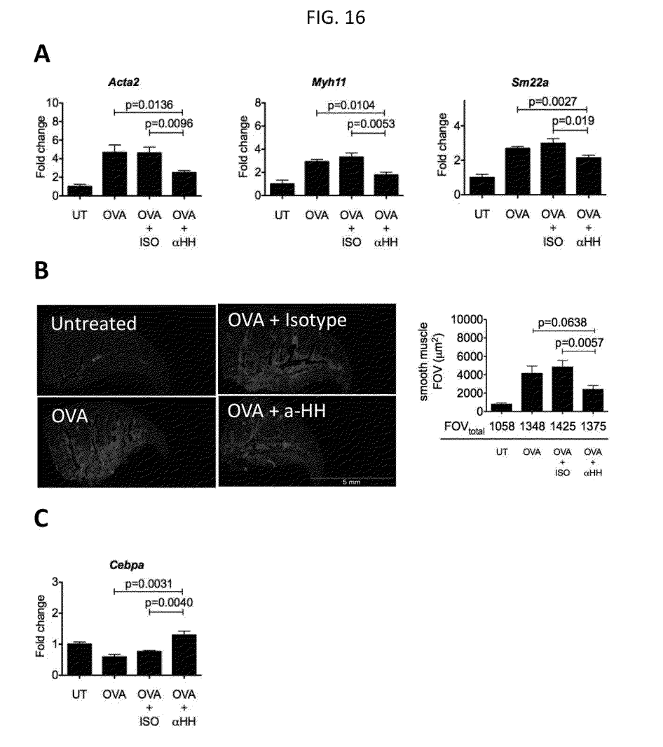

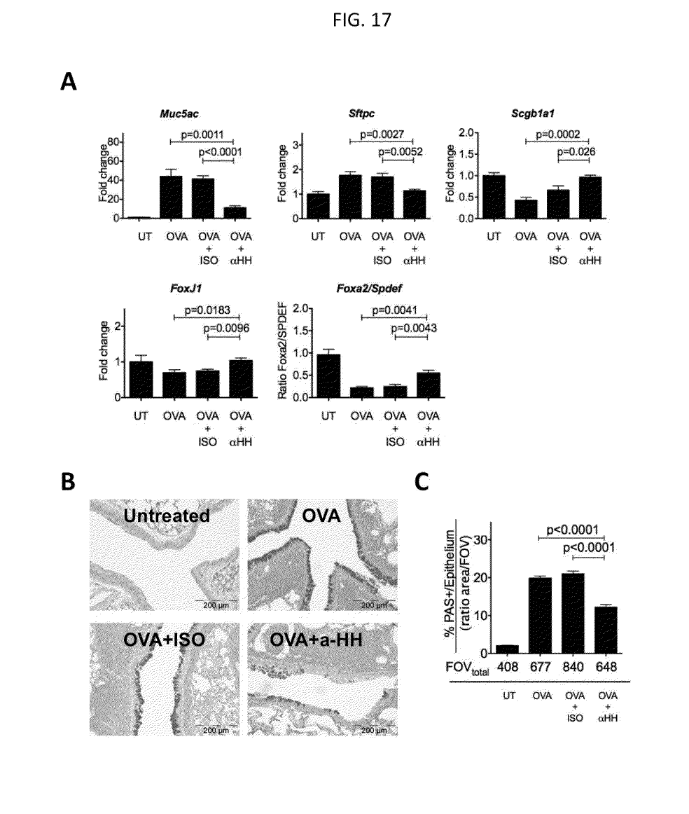

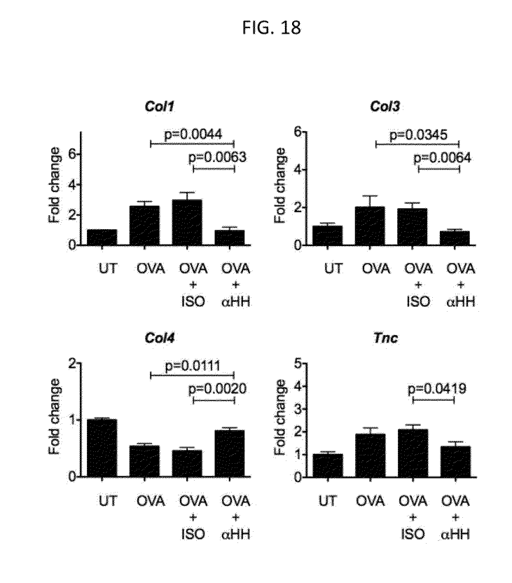

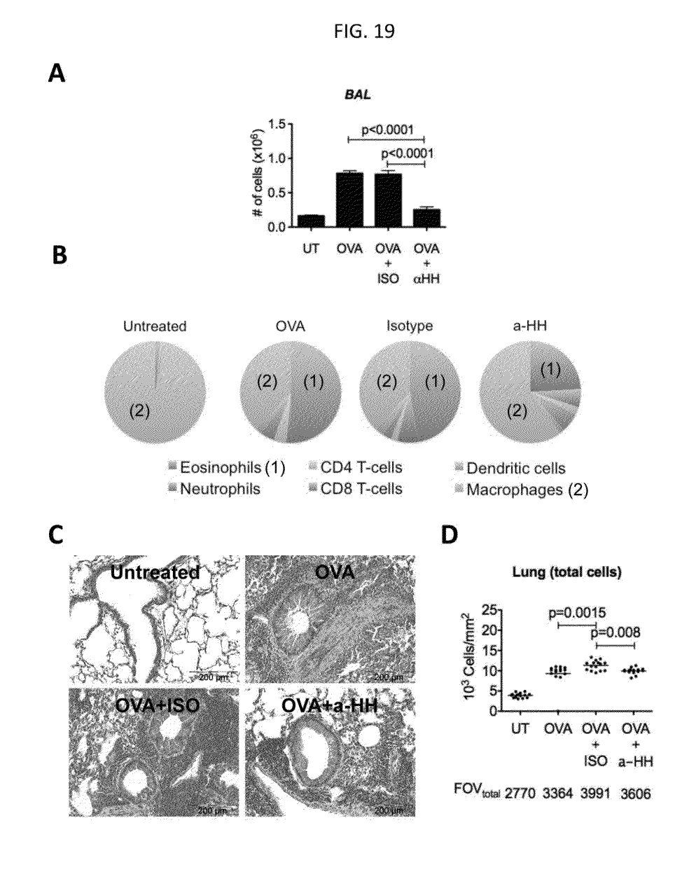

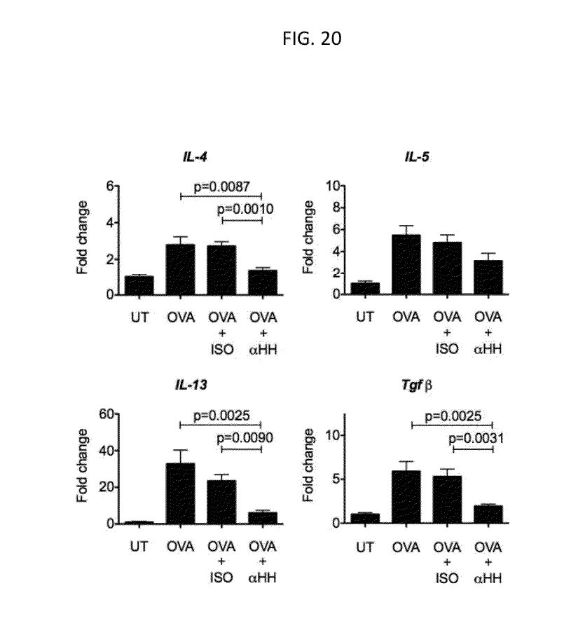

Moreover, treatment with anti-HH antibody 5E1 improved other parameters of tissue pathology and inflammation, resulting in a tendency to normalization of the expression of smooth muscle proteins genes and cebpa (FIG. 16); epithelial genes (FIG. 17); and ECM genes (FIG. 18). In addition, therapeutic treatment with anti-HH antibody of established asthma resulted in decreased cellular inflammation (FIG. 19) and decreased production of inflammatory cytokines (FIG. 20) in the lung.

Thus, inhibition of the HH pathway effectively improves lung function and reduces tissue pathology and inflammation in established experimental asthma. Moreover, since treatment with anti-HH antibody 5E1 resulted in increased expression of cebpa, the inhibition of the HH pathway may have the additional benefit of increasing the response to corticosteroid treatment in severe asthma.

### Example 3

### Increased Activation of the Hedgehog Pathway in Human Asthma

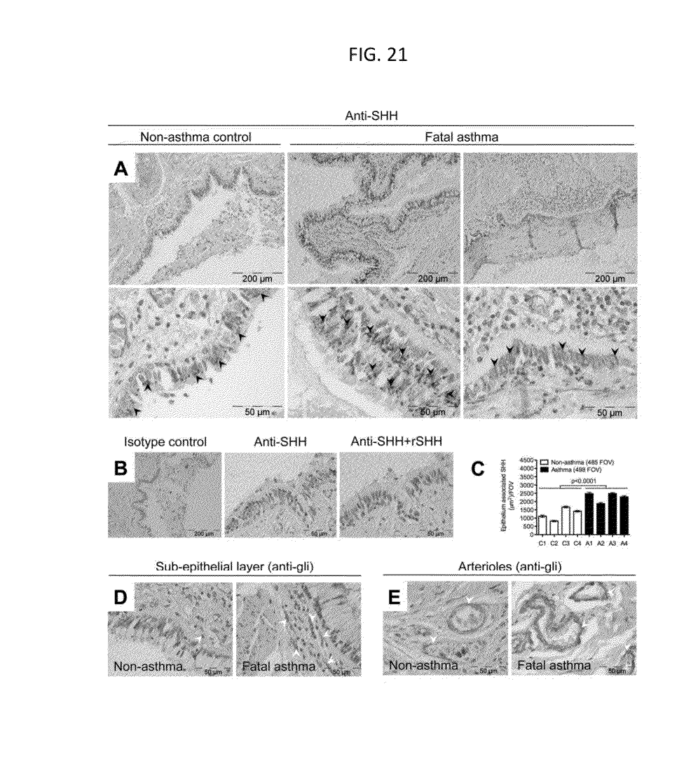

To determine whether the hedgehog pathway is activated in human asthma we analyzed the expression of SHH and GLI1 in sections of lung obtained from cases of fatal asthma (Ferreira, D. S et al. Toll-like receptors 2, 3 and 4 and thymic stromal lymphopoietin expression in fatal asthma. Clin Exp Allergy 42:1459-1471 (2012)). In non-asthmatic lung tissue, staining with anti-SHH antibody revealed a predominant sub-apical epithelial localization of SHH, whereas lateral and basal localization of SHH predominated in fatal asthma (FIG. 21), suggesting cellular translocation of SHH in asthma. Specificity of the staining was confirmed by using isotype controls or pre-absorbing the primary antibody with recombinant. SHH. The total amount of epithelium-associated SHH was 60% higher in asthma compared with non-asthmatic controls (FIG. 21). Consistent with these data, human lung sections stained with anti-GLI1 antibody revealed an increased frequency of GLI1⁺ sub-epithelial stromal cells and intense staining in vascular smooth muscle from asthmatic patients (FIG. 21). Together these data suggested increased activity of the HH pathway in human asthma.

### Materials and Methods

### Human Samples

Lung tissue was obtained at autopsy from 4 non smoking patients who died from an acute asthma exacerbation between 2005-2006 (Department of Pathology, Sao Paulo University). Control lung tissue was obtained from non-smoking subjects who died of non-pulmonary causes and with no previous history of asthma, wheeze or lung disease, and with no gross or microscopic lung pathology. These samples are from a previously described study population. Patient details are shown in table S1. The study was approved by the Human Studies Review Board of the Sao Paulo University Medical School (CAPPesq-HCFMUSP).

### Mice

A/J mice 6-8 weeks of age were used in all experiments. A/J mice we purchased from The Jackson Laboratories, USA, and bred and housed in the specific pathogen-free animal facility of the Biological Research Center (BRC), A*STAR, Singapore. All animal procedures were approved by the BRC/A*STAR Institutional Animal Care and Use Committee.

### Experimental Asthma and Treatment Regiments

Chronic allrgic inflammation was induced in A/J and BALB/c mice using minor modifications to published protocols (3). In brief, 100 μg chicken ovalbumin (OVA, endotoxin level 0.0435 EU/μg OVA as determined by LAL test; Sigma) was administered by intranasal route 3 times per week over 4-8 weeks with or without concurrent intraperitoneal (i.p.) injection of 250 μg aSHH monoclonal antibody (mAb) 5E1 (4) or an isotype-matched control mAb. For prophylactic studies, mAb treatment commenced at day-1 and continued for the duration of the experiment. For therapeutic studies, mAb treatment started 4 weeks after the first intranasal application of OVA and continued for another 4 weeks. For induction of chronic experimental asthma in BALE/c mice, 20 μg of Dermatophagoides pteronyssinus extract (house dust mite, HDM, Greer Laboratories, Lenoir, USA) dissolved in 40 μl PBS was given intranasal for 4 weeks. Antibodies were administered as described for A/J mice treated with OVA.

### Lung Function Measurements

Bronchial responsiveness was measured 24 h after OVA challenge as described elsewhere but with minor modifications. Briefly, mice were anaesthetized using a combination of ketamine, xylazine and acepromazine, and were then paralysed with pancuronium bromide. The trachea was exposed, cannulated and connected to a ventilator. Measurement of airways responsiveness to methacholine chloride (Mch) was performed using the flexiVent® system (SciReq Inc., Montreal, Canada). Mice were ventilated at a tidalvolume of 12 mL/kg and a respiratory rate of 150 breaths/min. The post-expiratory end pressure (PEEP) was maintained at 3.0 cm H₂O. The mice were ventilated for 2-5 min to establish a baseline before being nebulized with increasing doses of Mch (0-40 mg/ml). Animals were kept on a warming pad during the procedure. Respiratory mechanics were assessed using the linear first-order single compartment model (snapshot), which provides resistance of the total respiratory system (R) and compliance (C). For each dose of Mch nebulized, 20 alternating measurements of R (and C) were registered.

### BAL Collection and Analysis

BAL was collected as previously described. In brief, the trachea was cannulated and the lungs were rinsed with three times 0.8 ml of PBS containing 1 mM EDTA. BAL-fluid was separated from the cell fraction by centrifugation and

List of antibodies and application

| | | | |
|---|---|---|---|
| Target | Clone | Conjugate | vendor |

In vivo

| | | | |
|---|---|---|---|
| Shh | 5E1 | — | IOWA¹ |
| Isotype control | 13C4 | — | ATCC² |

Immunohistochemistry/Immunofluorescence

| | | | |
|---|---|---|---|
| Gli1 | Rb | — | Pierce³ |
| SHH (human) | Rb | — | Pierce³ |
| Smooth muscle | 1A4 | Cy3 | Sigma⁴ |
| Histone H3 (phopho | Rb | — | Abcam⁵ |
| 10) | polyclonal | | |
| Rabbit Ig (2^{nd} Ab) | polyclonal | HRP | Promega⁶ |

ELISA

| | | | |
|---|---|---|---|
| IgG1 | | HRP | Southern Biotech⁷ |
| IgE (capture) | | — | Invitrogen⁸ |
| IgE (detection) | | HRP | Southern Biotech⁷ |
| IL4 (capture) | 11B11 | — | eBioscience⁹ |
| IL4 (detection) | BCD6-24G2 | Biotin | eBioscience⁹ |
| TGFβ1-3 (capture) | 1D11 | — | R&D systems¹⁰ |
| TGFβ1 (detection) | IgY | Biotin | R&D systems¹⁰ |
| | Polyclon | | |

Ex vivo

| | | | |
|---|---|---|---|
| IL4 | 11B11 | — | Biolegend¹¹ |
| TGFβ | 1D11 | — | ATCC² |

Flowcytometr

| | | | |
|---|---|---|---|
| MHC-II.IEk | 14.4.4S | A647 | eBioscience⁹ |
| CD11c | N418 | PECy7 | eBioscience⁹ |
| CD11b | M1/70 | PE/PECy5.5 | eBioscience⁹ |
| Siglec-F | E50-2440 | PE | BD-Pharmingen¹² |
| GR-1 | RB6-8C5 | FITC | eBioscience⁹ |
| CD8α | 53-6.7 | PE | eBioscience⁹ |
| CD4 | RM4-5 | APC | eBioscience⁹ |
| CD19 | 1D3 | PECy7 | eBioscience⁹ |
| FceRI | MAR1 | FITC | eBioscience⁹ |
| cKit | ACK45 | PE | BD-Pharmingen¹² |
| CD49b | DX5 | FITC | eBioscience⁹ |

¹Developmental Studies Hybridoma Bank, University of Iowa, USA
²American Type Culture Collection, Manassas, VA, USA
³Thermo Fisher Scientific Inc., Pierce Protein Biology Products, Research Instruments, Singapore
⁴Sigma-Aldrich Pte. Ltd., Singapore
⁵Abcam plc., Abcell Pte. Ltd., Singapore
⁶Promega PTE LTD, branch office Singapore
⁷Southern Biotech, Scimed (Asia) Pte. Ltd., Singapore
⁸Invitrogen, Life Technologies Pte. Ltd., Singapore
⁹eBioscience Inc., Immunocell, Singapore
¹⁰R&D systems, Immunocell, Singapore
¹¹Biolegend, Genomax Tehnologies Pte, Ltd., Singapore
¹²BD Biosciences Pharmingen, Biomed Diagnostics Pte. Ltd, Singapore.

then stored at −80° C. The cellular composition of BAL was analyzed by flow cytometry (Facs Calibur, BD Biosciences). Antibodies are listed in the below table.

### Quantitative PCR Analysis of Gene Expression

Lungs were perfused with 1 mM EDTA in PBS to remove blood and the upper left lobe was collected in TRIzol (Invitrogen). The lung-draining lymph nodes (mediastinal and tracheobronchial) were collected in RNAlater (invitrogen). RNA extraction (TriZol, protocol Invitrogen), cDNA synthesis and quantitative PCR (Q-PCR, BioRad; Invitrogen Sybr green mastermix, 1 μM of both forward and reverse primers and 15-20 ng of template cDNA) were performed according to standard procedures and manufacturers protocols. Q-PCR reactions were carried out using a BioRad cFX96 RT-PCR instrument. Primers are listed in the table below. Gene expression was normalized to β-actin and results are expressed a fold-change in individual samples (relative to the average of the untreated samples in each experiment).

List of primers

| Gene | Forward (5′-3′ direction) | Reverse (3′-5′ direction) |
|---|---|---|
| Shh | AGGGGCCAGCGGCAGATATG | TTTGCACCTCTGAGTCATCAGCCG |
| (mouse | | |
| SHH | GGACAGGCTGATGACTCAGAGGT | ACGTGGTGATGTCCACTGCGC |
| (human | | |
| Dhh | AGCCGGATTCGACTGGGTCTAC | GGTCCAGGAAGAGCAGCACTG |
| Gli1 | GCGAAGCGTGGAGAGTCCGG | CTCAGCCACTCACCAGGAGGGA |
| Ptch1 | CCATACACCAGCCACAGCTTCG | GGAGGCTGGAGTCTGAGAACTG |
| Ptch2 | CCAGCAGCCAGCATGTAGTCAC | CTCGTGTCTGGAGCAGTAAAGG |
| IL4 | GACGCCATGCACGGAGATG | TGCGAAGCACCTTGGAAGC |
| IL5 | AGCAATGAGACGATGAGGC | ACACTTCTCTTTTTGGCGGT |
| IL13 | AGCATGGTATGGAGTGTGG | CCTCTGGGTCCTGTAGATG |
| IL33 | CAATGTTGACGACTCTGGAAA | GACTTGCAGGACAGGGAGAC |
| INFg | GCTTTGCAGCTCTTCCTAT | TTCCACATCTATGCCACTTG |
| Tgfb | CTGCTGACCCCCACTGATA | GCTGAATCGAAAGCCCTGTA |
| MucSac | GTGGTACGAGCCTTCAACCCAGG | ACTCCTGGACACGGCGTAGC |
| P63 | TCACGACCCAGGGGCTGACC | GCAGCTGCCTGTGGTCCAGG |
| Sftpc | TAGAAACCGCAGCGGGACAGG | CCTGGCCCGTAGGAGAGACACC |
| Scgb1a | GCGGGCACCCAGCTGAAGAG | GGAAGCCGAGGAGCCGAGGA |
| Foxa2 | TGCTGGGAGCCGTGAAGATGGAA | GCTCATGTTGCCGGAACCGCC |
| Spdef | AGAGGACCTCGCCTGGGACC | CGGAGCACGACGAGTCCACC |
| Foxj1 | CTTCCGCCATGCAGACCCCA | AGCAGGCGCTCTGCGTACTG |
| Cebpa | GCCGGCTACCTGGACGGCAG | TCCTCGCGGGGCTCTTGTTTG |
| Acta2 | GCTGTCAGGAACCCTGAGACGC | AGCATCATCACCAGCGAAGCCG |
| Myh11 | TGAGCCACCAGGAGAGGAAACGA | GTCTGAGTCCCGAGCGTCCAT |
| Sm22a | TGGTTTATGAAGAAAGCCCAGGAGC | GATGATCTGCCGGGGTCGCC |
| Tnc | CAGACAGACAACAGCATCAC | GACAGCAGAAACACCAATCC |
| Col1 | ACGGCTGCACGAGTCACA | GGCAGGCGGGAGGTCTT |
| Col3 | GTTCTAGAGGATGGCTGTACTAAACACA | TTGCCTTGCGTGTTTGATATTC |
| Co14 | ACGCTGTTGGTACAGCCGCC | CGACACCAGGCGCTCCCTTG |
| Actb | TGACAGGATGCAGAAGGAGA | GTACTTGCGCTCAGGAGGA |
| (mouse | | |
| ACTB | AACGGCTCCGGCATGTGCAA | CATCACGCCCTGGTGCCTGG |
| (hum | | |

### Cell Culture

Serial dilutions of BAL-fluid were added to mouse epithelial cell line LA4 with or without prior depletion of IL-4 and/or TGFβ. Cytokine depletion was achieved by three sequential incubations of BAL-fluid in plates coated with anti-IL-4 and/or anti-TGFβ. LA4 cells and human epithelial cell line A549 (ATCC) were treated with various concentrations of recombinant human TGFβ or recombinant mouse/human IL-4 (Peprotech). Cells were collected 24 h after stimulation, then lysed in TRIzol and processed for Q-PCR analysis of SHH expression.

### ELISA

OVA-specific IgG1 and IgE antibodies in serum were measured by ELISA. Determination of OVA-specific IgE was carried out following removal of IgG using GammaBind Plus Sepharose beads (GE Healthcare. Ltd, Sweden). ELISA plates were coated with 2 μg/ml OVA and the antibodies detected by addition of goat biotinylated anti-mouse IgG1 or rat anti-mouse IgE conjugated to HRP, respectively. Serum titer was defined as the reciprocal dilution at which 50% of maximum OD450 absorbance was observed. For OVA-specific IgE measurements, the samples were tested undiluted and OD450 values are shown. For the determination of total IgE, plates were first coated with 2 μg/ml anti-IgE capture antibody.

Matched antibody. ELISA pairs for IL-4 (eBioscience) and TGFβ1 (R&D systems) were used for evaluation of BAL-fluid. In the case of TGFβ1, the samples were first acidified by HCl treatment to activate latent TGFβ1.

### Histology, Immunofluorescence and Immunohistochemical Analyses

Paraffin-embedded lungs were cut at 4 μm thickness resulting in 5 sections per slide per mouse each 100 μm apart. All histological analyses were performed on at least 4 mice per group using samples from at least 2 independent experiments. Slides were de-parafinized with Histoclear (Sigma Life Science, Singapore) followed by rehydration. For antibody staining, antigen retrieval was performed in sodium citrate buffer (pH6) at 95° C. PAS, H&E, Hoechst 33342 and alpha smooth muscle actin (αSMA) slides were digitalized at 200× using a TissueFax slide scanner (TissueGnostics GmbH, Austria) yielding approximately 250-400 pictures (fields of view, FOV) per section. All other staining was manually assessed at either 100× or 400× magnification (Cell^{A}, Olympus CX31). Each figure shows the total number of images (FOV) analyzed. For analysis of epithelium (PAS, SHH), regions of interest were set around the epithelium and only images containing epithelium were included.

### Nuclear Staining

Harris Haematoxilin and Eosin Y (Sigma-Aldrich, Singapore), or nuclear dye Hoechst 33342 (Invitrogen) were used to stain nucleated cells. FOV were analyzed using TissueQuest or HistoQuest (TissueGnostics GmbH, Austria) for the quantification of cell staining.

### PAS Staining

PAS staining (PAS Stain kit, NovaUltra, IHCworld) was used to quantify total mucus production. Only sections containing epithelium were analyzed with TissueQuest. Regions of interest (ROI) were set around the bronchial epithelium and results for each ROI were expressed as the area of PAS+ staining per surface area of epithelium. Thresholds for background staining were determined by analyzing 100 FOV in which no visible PAS staining was observed (310 μm² total PAS+staining/ROI for the 8 weeks prophylactic treatment, and 107 μm² for the 4 weeks OVA followed by 4 weeks antibody treatment).

### Picro-Sirius Red Staining

For quantification of collagen, slides were stained using the picro-sirius red method. Direct Red 80 (Sigma) was dissolved 0.1% w/v in saturated picric acid solution (Sigma) and slides were stained for 2 h. Sections were recorded at 100× and analyzed with TissueQuest. Data are expressed as relative amount of collagen/total lung area.

### Antibody Staining

Staining and quantification of αSMA in lung sections was analyzed using HistoQuest and expressed as total amount of αSMA staining per section. A threshold value of 50 μm² positive staining/section was applied to compensate for background. Both SHH and GLI1 primary staining were performed overnight at 4° C. using unconjugated antibodies. Incubation with secondary biotinylated antibodies was conducted for 3 h at RT. Detection with SA-HRP was carried out using the DAB-kit (Invitrogen) for 10 min at RT. For staining controls the primary antibody was replaced with rat-IgG or rabbit serum. Human SHH staining specificity was determined by pre-incubating the primary anti-SHH antibody with recombinant human SHH (eBioscience). Sections were recorded at 400× magnification and analyzed with TissueQuest.

### Statistics

One-way ANOVA was performed using GraphPad Prism v5.0b for Macintosh, (GraphPad Software, USA). To obtain exact p-values, groups that showed significant difference by Bonferroni's post-test were subjected to an unpaired two-tailed student's t-test. T-tests were used for all comparisons between two groups

### Applications

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of treating a disease or disorder characterised by one or more of hypersecretion of mucus, epithelial cell hyperplasia, smooth muscle hypertrophy, fibrosis, inflammation, bronchial hyper-responsiveness or decreased lung function comprising administering a 5E1 monoclonal antibody or a monoclonal antibody which binds the same epitope as 5E1 or a functional antigen binding fragment thereof to a patient in need thereof.

2. The method according to claim 1, wherein the disease or disorder is a respiratory disease selected from the group consisting of asthma, chronic obstructive pulmonary disease, bronchiolitis obliterans, chronic bronchitis, pulmonary fibrosis and cystic fibrosis.

3. The method according to claim 1, wherein the disease or disorder is a gastrointestinal or reproductive disease or disorder characterised by one or more of hypersecretion of mucus, epithelial cell hyperplasia, smooth muscle hypertrophy, fibrosis or inflammation.

4. The method according to claim 2, wherein the respiratory disease is asthma.

5. The method according to claim 1, wherein the route of administration is selected from the group consisting of oral administration, intravenous administration, parenteral administration and local administration in the airways.

6. The method according to claim 1, wherein said 5E1 monoclonal antibody or said monoclonal antibody which binds the same epitope as 5E1 or a functional antigen binding fragment thereof is administered with one or more further therapeutic agents.

7. The method according to claim 6, wherein the one or more further therapeutic agents are selected from the group consisting of short acting beta-adrenoceptor agonists, anticholinergic agents, adrenergic agonists, corticosteroids, long acting beta-adrenoceptor agonists, leukotriene antagonists, an antagonist of Smoothened, an antagonist of Smoothened activation, an antagonist of Gli, anti-IgE antibodies or compounds, anti-cytokine antibodies or compounds and mast cell stabilisers.

8. The method according to claim 7, wherein the antagonist of Smoothened is selected from the group consisting of cyclopamine or derivatives thereof, vismodegib, IPI-926, LDE225, XL139 and PF-0449913.

9. The method according to claim 6, wherein the monoclonal antibody and the one or more further therapeutic agents are administered sequentially, simultaneously or separately.

10. The method according to claim 1, wherein said treatment restores C/EBPα levels in the lung to improve a response to corticosteroid treatment in asthma.

11. A kit when used in accordance with the method of claim 1 comprising a 5E1 monoclonal antibody or a monoclonal antibody which binds the same epitope as 5E1 or a functional antigen binding fragment thereof together with instructions for use.

12-21. (canceled)

22. The method according to claim 1, wherein said treatment increases the expression of C/EBPα in the lung to enhance a response to corticosteroid treatment for asthma.

23. (canceled)
